(19) United States
(12) Patent Application Publication
(10) Pub. No.: US 2023/0390323 A1
(43) Pub. Date: Dec. 7, 2023

(54) SPRAYABLE ANTIVIRAL FORMULATION

(71) Applicant: The University of Birmingham, Birmingham (GB)

(72) Inventors: Liam Michael Grover, Birmingham (GB); Richard John Asa Moakes, Birmingham (GB)

(21) Appl. No.: 18/033,160

(22) PCT Filed: Oct. 22, 2021
(86) PCT No.: PCT/EP2021/079402

§ 371 Date: Apr. 21, 2023

(30) Foreign Application Priority Data
Oct. 22, 2020 (GB) 2016771.4

(51) Int. Cl. A61K 31/737; A01N 25/06; A01N 43/16; A61K 31/727; A61K 9/00; A61P 31/14; A01P 1/00

(52) U.S. Cl. CPC A61K 31/737; A01N 25/06; A01N 43/16; A61K 31/727; A61K 9/0043; A61P 31/14; A01P 1/00

(57) ABSTRACT

The present invention relates to a sprayable antiviral formulation comprising an antiviral agent and a carrier polymer, wherein the antiviral agent comprises a sulphated polysaccharide and the carrier polymer comprises a non-sulphated polysaccharide. In particular, the present invention relates to a sprayable antiviral formulation which can be used as a nasal spray or as a multi-surface spray.

### TECHNICAL FIELD

The present invention relates to a sprayable antiviral formulation comprising an antiviral agent and a carrier polymer, wherein the antiviral agent comprises a sulphated polysaccharide and the carrier polymer comprises a non-sulphated polysaccharide. In particular, the present invention relates to a sprayable antiviral formulation, which can be used as a nasal spray or as a multi-surface spray.

### BACKGROUND

Transmission of viruses may occur through four routes: direct contact, via physical contact with a carrier; indirect contact, via interactions with contaminated objects; droplet and airborne transmission, often through coughs, sneezes and breathing; and aerosolization, i.e. atomised virus suspended in airflow.

Airborne transmission of respiratory pathogens (whether through droplets or atomisation) is particularly deleterious, with the virus effectively locally delivered to the respiratory pathways. There are many airborne viruses including: influenza-, rhino-, adreno-, entero- and corona-virus. The latter, coronaviridae (CoVs) family, are implicated in a variety of gastrointestinal, central nervous system and respiratory diseases (MERS, SARS); with the latest strain, SARS-CoV-2, receiving much attention due to its devastating impact within the 2020 pandemic. SARS-CoV-2, like all coronaviruses, contains large positive-strand RNA genomes packed within a helical capsid, all housed within a phospholipid bilayer envelope formed on budding. Associated with the viral membrane are three main proteins: membrane and envelope proteins (associated with assembly), and spike proteins. The spike proteins, which give rise to its corona shape, are essential for virus survival, mediating entry to the host cell. Additionally, the protein also plays a crucial role in determining host range and tissue tropism, alongside being responsible for inducing many of the host immune responses. To date, facilitation of viral entry into a host cell is believed to arise through specific motifs within the spike protein, which strongly interact with ACE2 receptors. ACE2 is known for its role in regulating oxygen/carbon dioxide transfer, commonly found within the respiratory epithelia. In particular SARS-CoV-2 has been found to target the ciliated and goblet cells, where subsequent viral shedding results in extensive viral loads, especially within the upper respiratory tract.

Respired air is primarily routed through the nose. Even though the nasal passages present the highest resistance to airflow, on average ca. 10,000 L of air is inhaled by a healthy human per day. Only once this pathway becomes overloaded does the body switch to respiratory through the mouth. For this reason, the nasal cavity supports two major roles: climate control, creating the correct levels of humidity and air temperature; and removal of foreign particles, including dust, airborne droplets and pathogens. Anatomically, the nose consists of two cavities roughly 10-15 cm in length and 5 cm in height, producing a total surface area of about 150 cm². Inspired air flows up through the nasal vestibule (nostril) and passes through the slit-like meatus structures (inferior, middle and superior) and back through the nasopharynx. At a cellular level the majority of the cavity consists of a typical airway epithelium, comprising of four main cell types: basal, ciliated/non-ciliated columnar and goblet cells. The columnar cells, whether ciliated or not, are coated by microvilli. Their role, to prevent drying, supports the cilia in performing mucociliary clearance of mucins produced in the goblet cells. Additionally, the presence of cilia and microvilli drastically increases the effective surface area (ca. 9.6 m²), providing a highly efficient platform for filtration. Unfortunately, such large surface areas also provide greater exposure in terms of viral entry.

The airborne risk imposed not only through ventilation systems and crowds, but re-suspension of the virus from inanimate objects, including personal protective equipment, vociferates the need for new and novel devices that not only prevent contraction, but stop spread thereafter.

The present invention has been developed with these issues in mind.

### SUMMARY OF THE INVENTION

According to a first aspect of the invention, there is provided a sprayable antiviral formulation comprising an antiviral agent and a carrier polymer. The antiviral agent comprises a sulphated polysaccharide and the carrier polymer comprises a non-sulphated polysaccharide.

In use, the formulation forms a coating layer on the surface, which it is applied to. For the purposes of the present invention, it is important that the formulation is sprayable rather than jettable to ensure adequate coating coverage when the formulation is applied to a surface, e.g. a non-anatomical surface such as a countertop or an anatomical surface such as an oral or nasal cavity. Uniformly coating a nasal cavity is particularly challenging, due to the relatively large surface area and difficulty of access. When used as a nasal spray, a jettable formulation not only provides poor coverage but can also cause irritation on contact with the nasal wall, negatively affecting patient compliance. The inventors of the present invention have found that a sprayable antiviral formulation, which contains an antiviral agent comprising a sulphated polysaccharide and a carrier polymer comprising a non-sulphated polysaccharide achieves a good coating coverage when sprayed onto a surface, including a surface within a nasal cavity.

Sprayability

The term “sprayable” as used herein is intended to mean that the formulation produces a plume of droplets rather than a jet of fluid when sprayed from a normal spray nozzle, whereas a formulation which forms a jet when sprayed may be considered to be “jettable”. A jet can be considered to be a coherent stream of fluid, whereas a plume is formed when a jet disrupts to form small, discrete droplets. The jet may disrupt to form a plume of droplets either immediately after ejection from the nozzle, or at a distance away from the nozzle.

For the purposes of the present disclosure, a formulation may be deemed to be sprayable if the jet disrupts to form a plume of droplets within 0-5 cm of the nozzle when sprayed. Without wishing to be bound by theory, it is thought that a formulation which forms a plume within 0-5 cm of the nozzle when sprayed creates a sufficient distribution of droplets to coat a mucosal surface within a confined interior space, such as an oral or nasal cavity, as well as providing good coverage when sprayed onto a larger external surface, such as a countertop. In some embodiments, where the formulation is intended for use as an oral or nasal spray, the formulation may form a plume within 0-2.5 cm of the nozzle when sprayed.

In some embodiments, “sprayability” may be assessed by measuring the percentage coverage of a set area that the formulation is sprayed onto from a set distance. In some embodiments, the percentage coverage is determined using the following procedure:
10 mL formulation is mixed thoroughly with 0.1 mL black ink in a falcon tube;
the cap of the falcon tube is replaced with a normal spray applicator (nozzle aperture 750 μm);
the applicator is primed by depressing the manual actuator 10 times;
the formulation is sprayed vertically upwards, once, onto a horizontal sheet of paper suspended 10 cm away from the spray applicator nozzle;
the paper is then allowed to dry before scanning at 600 dpi in greyscale;
the image is cropped to a size of 2000×2000 pixels around the spray distribution, using image analysis software such as ImageJ; and
the image analysis software is used to analyse the percentage of the 2000×2000 pixel area covered by the spray, for example by using the “Analyse Particles” function in ImageJ.

In some embodiments, the formulation is deemed to be “sprayable” if the percentage coverage, as determined by the above procedure, is at least 13%. In some embodiments, the percentage coverage achieved by the formulation may be at least 13%, at least 15%, at least 20%, or at least 25%. In general, the higher percentage coverage achieved by a formulation, the more sprayable the formulation may be deemed to be.

A scale bar may be applied within the image analysis software to determine the number of pixels that corresponds to 1 cm in the 2000×2000 pixel cropped image, such that the percentage coverage can be converted to an area in cm². From this, the spray distribution radius can be calculated using Equation 1:

$$\sqrt{\frac{area}{π}} = r \qquad [ 1 ]$$

Using the procedure above, a formulation may be deemed to be “sprayable” if the distribution radius is at least 1.8 cm. In some embodiments, the distribution radius achieved by the formulation may be at least 1.8 cm, at least 2.0 cm, at least 2.2 cm, or at least 2.4 cm. In general, the higher the distribution radius, the more sprayable a formulation may be deemed to be.

The distribution radius and the distance that the nozzle is held from the paper when sprayed can in turn be used to determine the spray angle, i.e. the maximum angle at which droplets in the plume deviate away from a central axis along which the formulation is sprayed, using Equation 2 (where adj is the distance that the nozzle is held from the paper when sprayed and opp is the distribution radius):

$$x ° = {tan}^{- 1} ( \frac{opp}{adj} ) \qquad [ 2 ]$$

Using the procedure above, a formulation may be deemed to be “sprayable” if the spray angle is at least 10°. In some embodiments, the spray angle achieved by the formulation may be at least 10°, at least 11°, at least 12°, at least 13°, or at least 14°. In general, the higher the spray angle, the more sprayable a formulation may be deemed to be. It will be understood that, in embodiments where the jet disrupts into a plume of droplets at a distance away from the nozzle rather than immediately after ejection, the actual angle formed by the plume away from the central axis of the jet may be higher than the angle calculated from the nozzle. However, for the purposes of the present disclosure, the effective spray angle is deemed to be the angle calculated from the nozzle to the surface that the formulation is sprayed on, as if the jet had disrupted into a plume of droplets immediately upon ejection from the nozzle.

Formulation

In some embodiments, the formulation is for use as a multi-surface spray. In use, the multi-surface spray may be sprayed onto any surface, such as a countertop, an external surface of an apparatus or article of personal protective equipment, or an internal surface of a tube for a ventilator, for example. After use, the multi-surface spray may be safely wiped or washed away.

In some embodiments, the formulation is for application on a mucosal surface. In some embodiments, the formulation is for use as an oral spray and/or a nasal spray. It will be understood that a formulation for use as an oral or nasal spray must be non-toxic and safe for use on mucosal surfaces. As such, it is preferred that the formulation is free or substantially free of oxidising agents commonly used for their antiviral properties, such as hydrogen peroxide, which may irritate the delicate mucosal membrane. After use, the spray may be removed by the body's natural mucus-clearing processes or manually by the user, e.g. washing an oral spray into the oesophagus by drinking a liquid such as water, or blowing the nose to remove a nasal spray.

In some embodiments, the formulation is free or substantially free of oxidising agents, such as hydrogen peroxide. In some embodiments, the formulation comprises less than 0.01% w/v oxidising agent.

The sprayable formulation of the present invention comprises a sulphated polysaccharide as an antiviral agent. Sulphated polysaccharides have been found to exhibit antiviral activity against a range of airborne viruses, including influenza-, rhino-, adeno-, entero- and coronaviruses. The antiviral activity of the sulphated polysaccharide in the claimed formulation helps to inhibit these viruses. The inventors of the present invention have also found that sulphated polysaccharides bind very effectively to airborne viruses, in particular coronaviruses such as SARS-CoV-2, thereby confining virus particles inside the formulation and preventing their further spread. The formulation of the present invention therefore has a dual-action prophylactic effect to prevent infection and/or transmission of airborne viruses when sprayed onto a surface, by providing a physical barrier which traps virus particles and prevents them from passing through the sprayed layer, and by having an antiviral effect against the virus once it is caught in the sprayed layer.

In some embodiments, the sprayable antiviral formulation is for use in the prevention of infection and/or transmission of an airborne virus. In some embodiments, the airborne virus is selected from one or more of influenza-, rhino-, adeno-, entero- and coronavirus. In some embodiments, the sprayable antiviral formulation is for use in the prevention of infection and/or transmission of a coronavirus. In some embodiments, the coronavirus is SARS-CoV-2.

Sulphated polysaccharides are polysaccharides in which a number of hydroxyl functional groups have been replaced by sulphate functional groups. Sulphated polysaccharides may be produced synthetically, for example by chemically modifying a polysaccharide, or naturally, for example in certain types of seaweed or algae. Non-limiting examples of sulphated polysaccharides which are suitable for use with the present invention include carrageenans, fucoidans, ulvans and heparin sulphate.

For embodiments of the present invention which are intended for application on mucosal surfaces, in particular, it is important that the formulation comprises a sulphated polysaccharide which interacts favourably with the mucous membrane and thus has good mucoadhesion. Without wishing to be bound by theory, it is thought that sulphated polysaccharides having good mucoadhesion may help to improve the longevity of the formulation on a mucosal surface, such as in an oral or nasal cavity, thereby extending the prophylactic effect against airborne viruses. Accordingly, in some embodiments, the formulation comprises a mucoadhesive sulphated polysaccharide.

Carrageenans are an example of naturally-occurring sulphated polysaccharides, extracted from red seaweed. Several different types of carrageenan exist, having different levels of sulphation, the most common being kappa-, iota- and lambda-carrageenan. Of these three types, kappa-carrageenan is the least sulphated, having one sulphate group per galactose repeat unit; iota-carrageenan has two sulphate groups per galactose repeat unit; and lambda-carrageenan is the most sulphated, having three sulphate groups per galactose repeat unit. The inventors of the present invention have found that carrageenans not only exhibit good mucoadhesion, but also bind very effectively to airborne viruses such as coronaviruses, in particular SARS-CoV-2.

In some embodiments of the present invention, the sulphated polysaccharide comprises a carrageenan. In some embodiments, the carrageenan is selected from the group consisting of lambda-carrageenan, iota-carrageenan and kappa-carrageenan. In some embodiments, the carrageenan is lambda-carrageenan.

Without wishing to be bound by theory, it is thought that the mucoadhesive properties of the sulphated polysaccharide are not the only factor which may affect the longevity of the sprayed formulation on a surface. For example, it is preferable that the formulation does not simply flow off the surface under its own mass, particularly on inclined or inverted surfaces, and so the viscosity of the formulation may also have an effect on retention. A formulation having higher viscosity may have higher retention on a surface. However, a formulation having higher viscosity may also be less sprayable in some cases.

In some embodiments, the formulation has a dynamic viscosity of 0.05 to 100 Pa·s at 25° C. It will be understood that individual components within the formulation might have a different inherent viscosity, but the dynamic viscosity of the formulation as a whole will be from 0.05 to 100 Pa·s, in some embodiments. Without wishing to be bound by theory, it is thought that in some embodiments the overall viscosity of the formulation may be affected by viscosity-modifying interactions between the individual components of the formulation as well as factors such as the concentration of individual components and overall dilution level. In some embodiments, the formulation has a dynamic viscosity of 0.05 to 50 Pa·s, 0.05 to 10 Pa·s, 0.1 to 10 Pa·s, 0.1 to 5 Pa·s, or to 2 Pa·s at 25° C.

The formulation of the present invention further comprises a carrier polymer. The carrier polymer comprises a non-sulphated polysaccharide. Non-limiting examples of non-sulphated polysaccharides suitable for use in the present invention include gellan, dextran, alginate, pectin and xanthan. In some embodiments, the carrier polymer comprises a mucoadhesive non-sulphated polysaccharide. In some embodiments, the carrier polymer comprises gellan. The inventors of the present invention have found that gellan provides particularly good sprayability and retention characteristics in conjunction with a sulphated polysaccharide.

The carrier polymer may be used to modify the total polymer content of the formulation, which may help to enhance the uniformity and thickness of the coating layer formed by the formulation when sprayed, thereby improving the effectiveness of the physical barrier provided by the sprayed formulation. The carrier polymer may also be used to modify related characteristics such as viscosity, sprayability and/or retention in embodiments where the sulphated polysaccharide alone does not provide the desired characteristics.

For example, some embodiments comprising moderate concentrations of iota-carrageenan as a sulphated polysaccharide, e.g. from 0.2 to 0.6% w/v, were found to have a viscosity of around 0.1-5 Pa·s and exhibited good retention and sprayability, so did not require a significant proportion of carrier polymer. On the other hand, some embodiments comprising higher concentrations of sulphated polysaccharide, e.g. over 1.0% w/v, or comprising sulphated polysaccharides having a higher degree of sulphation, such as lambda-carrageenan may require a higher proportion of carrier polymer to improve sprayability. A higher proportion of carrier polymer may also be added to some embodiments comprising a very low concentration of sulphated polysaccharide, in order to increase the total polymer content of the formulation.

Without wishing to be bound by theory, it is thought that the sprayability of the formulation could be partly affected by the degree of sulphation of the sulphated polysaccharide as well as the concentration and total polymer content, since polysaccharides having a moderate degree of sulphation (e.g. iota-carrageenan) were found to be sprayable at higher concentrations than polysaccharides having a higher degree of sulphation (e.g. lambda-carrageenan). However, the relationship between concentration and sprayability was found to be unrelated to the inherent viscosity of the sulphated polysaccharide. For example, lambda-carrageenan has a lower inherent viscosity than iota-carrageenan and was therefore predicted to be easier to spray. However, it was found that formulations comprising lambda-carrageenan were, in fact, less sprayable than iota-carrageenan and thus required addition of a carrier polymer to achieve good sprayability. Without wishing to be bound by theory, it is thought that the sprayability of the formulation could potentially be more correlated with the surface tension of the sulphated polysaccharide than its inherent viscosity, and that the carrier polymer may interact with the sulphated polysaccharide to modify the surface tension, resulting in improved sprayability.

In some embodiments, the sprayable antiviral formulation comprises a diluent. The diluent may be used to dilute the antiviral agent, carrier polymer and other components in the formulation to a desired concentration, to achieve a desired level of viscosity, and/or to achieve a desired level of sprayability. The diluent may be any solvent suitable for medical use. In some embodiments, the diluent is a saline solution. For example, the saline solution may be a phosphate-buffered saline solution.

In some embodiments, the concentration of antiviral agent in the formulation is from 0.1 to 1.0% w/v, based on the total volume of the formulation. In some embodiments, the concentration of antiviral agent in the formulation is from 0.1 to 0.9% w/v, from 0.1 to 0.8% w/v, from 0.1 to 0.7% w/v, from 0.1 to 0.6% w/v, or from 0.1 to 0.5% w/v, based on the total volume of the formulation.

In some embodiments, the formulation has a total polymer concentration consisting of, or consisting essentially of, the concentration of antiviral agent and the concentration of carrier polymer. In some embodiments, the total polymer concentration is from 0.1 to 2.0% w/v, from 0.1 to 1.5% w/v, from 0.1 to 1.0% w/v, or from 0.1 to 0.5% w/v, based on the total volume of the formulation. In general, a moderate to high total polymer content (e.g. greater than or equal to 0.4% w/v) may be desirable in order to provide a sufficiently thick and uniform layer when the formulation is sprayed onto a surface, although this must be balanced with other requirements such as sprayability, which may decrease with increasing polymer content.

In some embodiments, the ratio of antiviral agent to carrier polymer is from 90:10 to 10:90, from 75:25 to 10:90, from 75:25 to 25:75, or from 10:90 to 50:50 antiviral agent:carrier polymer. In some embodiments, the ratio of antiviral agent to carrier polymer is 25:75 antiviral agent:carrier polymer. In general, the ratio of antiviral agent to carrier polymer may be higher in embodiments where the total polymer concentration is lower, but it will be understood that the ratio of antiviral agent to carrier polymer and the total polymer concentration may be tailored together to achieve the desired viscosity, retention and sprayability characteristics in the formulation.

In some embodiments, the formulation further comprises a dispersing agent. The dispersing agent may help to ensure that the antiviral agent and other components, such as carrier polymer, are homogenously dispersed throughout the formulation. In some embodiments, the dispersing agent is one or more surfactants and/or salts. Examples of surfactants include phospholipids such as glycerophospholipids (e.g. lecithin), sorbitan esters, Tween 20-80, sucrose monostrate, sodium dodecyl sulphate, polysorbates and potassium sorbate. Examples of salts include monovalent salts such as those comprising sodium (e.g. NaCl) or potassium (e.g. KCl) and divalent salts such as those comprising calcium (e.g. CaCl₂)) or magnesium (e.g. MgSO₄).

According to a second aspect of the invention, there is provided a spray device comprising the formulation of the first aspect, a body for containing the formulation therein, and a nozzle for spraying the formulation. In some embodiments, the spray device is a nasal spray device. In some embodiments, the spray device comprises a pump for ejecting the formulation from the device through the nozzle. Alternatively, the spray device may be configured so that the formulation is ejected through the nozzle when manual pressure is applied to the device, for example by squeezing the body. The construction of the spray device may be in accordance with any known spray device, and suitable constructions will be known to the person skilled in the art.

According to a third aspect of the invention, there is provided a nasal spray formulation comprising an antiviral agent and a carrier polymer. The antiviral agent comprises a sulphated polysaccharide and the carrier polymer comprises a non-sulphated polysaccharide.

It will be understood that any of the features described in relation to the first aspect may apply equally to the third aspect, e.g. the types and concentrations of sulphated polysaccharide and carrier polymer, other components in the formulation, etc.

The formulations as described herein may find use in methods of prevention and/or treatment of airborne viruses, such as coronavirus.

### BRIEF DESCRIPTION OF THE FIGURES

FIG. 1 shows schematic diagrams showing a proposed mechanism of action for formulations according to the present invention in a nasal cavity;

FIG. 2 is a schematic representation of a nasal spray, in use;

FIG. 3 shows results of sprayability screening for a range of biopolymers;

FIG. 4 shows viscosity data for a range of biopolymers and mixtures thereof;

FIG. 5 shows viscosity data for mixtures of gellan and lambda-carrageenan;

FIG. 6 shows sprayability results for formulations comprising gellan and lambda-carrageenan;

FIG. 7 shows sprayability results for formulations comprising iota-carrageenan and a mixture of iota-carrageenan and gellan;

FIG. 8 shows droplet distribution data for formulations comprising gellan and lambda-carrageenan;

FIG. 9 shows spray coverage data for formulations comprising gellan and lambda-carrageenan;

FIG. 10 shows in vitro data demonstrating cell compliance and infection suppression for formulations comprising gellan and lambda-carrageenan;

FIG. 11 shows Hoechst stained images demonstrating infection suppression for formulations comprising gellan and lambda-carrageenan;

FIGS. 12 (a) and (b) show in vitro data demonstrating infection suppression for formulations comprising kappa-, iota- and lambda-carrageenan and FIG. 12 (c) shows the percentage of cells infected with SARS-CoV-2 when treated with various biopolymer mixtures: Gellan-Dextran Sulphate (DS), Gellan-Dextran (D), Gellan-Heparan Sulphate (HS);

FIG. 13 shows cell binding results for gellan, iota-carrageenan and lambda-carrageenan;

FIG. 14 is a plot showing the percentage of cells infected with SARS-CoV-2 as a function of treatment with gellan and gellan-sulphated polymer composite; and

FIG. 15 shows viral transduction as a function of various treatment groups (typical images used to determine degree of transfection).

### DETAILED DESCRIPTION

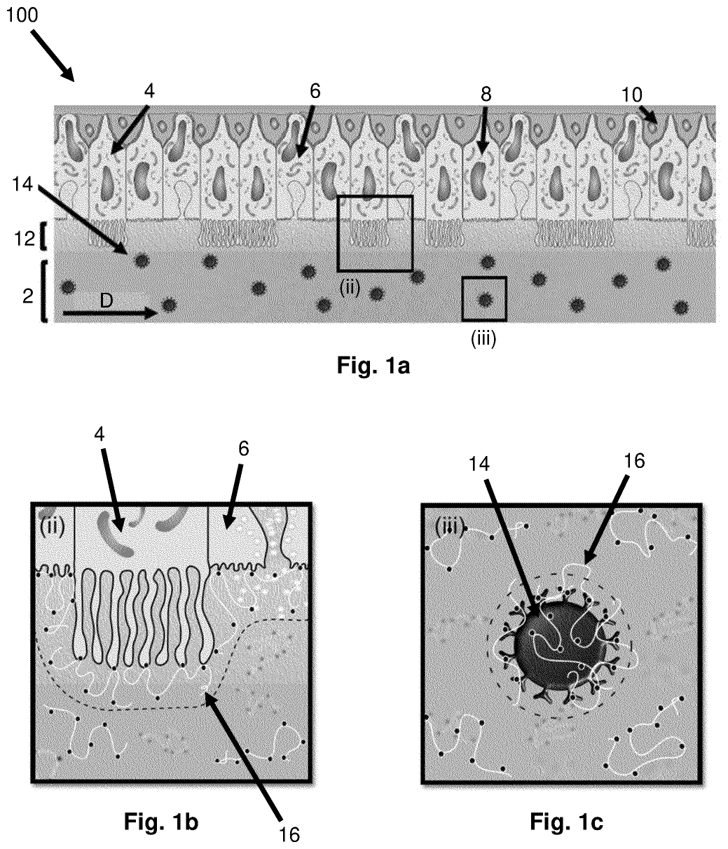

FIGS. 1 a-c show a proposed mechanism for how a formulation 2 according to the present invention may effect inhibition of SARS-Cov-2 in a nasal cavity. FIG. 1a shows a nasal epithelium 100 coated in a layer of nasal spray formulation 2. The nasal epithelium 100 comprises ciliated cells 4, goblet cells 6, non-ciliated cells 8 and basal cells 10. Goblet cells 6 produce a mucus layer 12, which is cleared over time by ciliated cells 4 to drain into the throat in the direction of drainage D. Ciliated cells 4 and goblet cells 6 contain a high number of ACE2 receptors and thus are particularly vulnerable to infection by SARS-Cov-2.

A formulation according to an embodiment of the present invention is sprayed into the nasal cavity, forming a formulation layer 2 which adheres to the mucus layer 12 and provides a physical barrier against the virus, trapping virus particles 14 within the formulation layer 2 and preventing them from infecting the ciliated cells 4 and goblet cells 6. The formulation layer 2 may be naturally cleared to drainage along with the mucus layer 12 or expelled by blowing the nose, safely removing the trapped virus particles 14 from the nasal cavity.

As shown in FIG. 1b, the polymer 16 contained within the formulation 2 may create a steric barrier across the cell interface, thereby blocking virus particles 14 from entering the cells 4, 6. As shown in FIG. 1c, the polymer 16 may also create a steric barrier around the interface of the virus particles 14, thereby preventing the virus from entering the cells 4, 6. In addition, the polymer in the formulation of the present invention comprises sulphated polysaccharide, which has been shown to have antiviral activity and thus may deactivate virus particles trapped within the formulation.

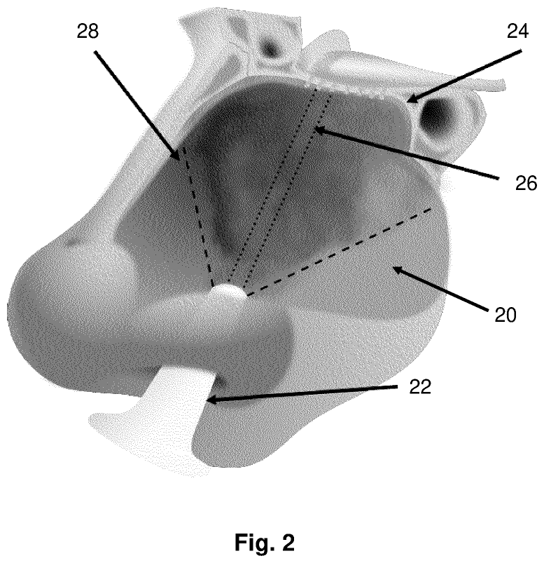

FIG. 2 shows a schematic diagram of a nasal spray formulation being sprayed into the nasal cavity 20 of a user. In use, the nozzle 22 of the spray device is inserted into the nostril and actuated, e.g. by squeezing the body of the spray device or by operating a pump in the spray device. The formulation is ejected from the spray nozzle 22 into the nasal cavity 20 and coats the nasal epithelium 24 at the back of the nasal cavity 20.

Providing a prophylactic coating across the entire nasal epithelium poses a significant challenge, due to the difficulty of access via the nostrils, and the relatively large surface area and complex topology of the nasal epithelium (including inclined surfaces and ceilings). The formulation of the present invention has therefore been engineered to provide good sprayability and retention on inclined surfaces.

In a jettable formulation, the formulation is ejected from the nozzle 22 in a continuous stream or “jet” 26 along the central axis of the spray and thus only coats the nasal epithelium 24 in a concentrated location. In a sprayable formulation, in accordance with the present invention, the formulation forms a “plume” 28 of droplets, which spreads away from the central axis of the spray and coats a larger area of the nasal epithelium 24.

Initial Screening

In order for the illustrated trapping mechanism to work in practice, it is important that the formulation is retained on the application surface for a reasonable amount of time and does not flow off the surface under its own mass. Preferably, the formulation should be retained on a surface for as long as possible, to maximise the duration of the prophylactic effect against airborne viruses.

It is also important that the formulation is sprayable, in order to provide sufficient, uniform coverage over as much of the area of the surface as possible.

The inventors of the present invention therefore began by screening a number of different biopolymers for their retention and sprayability characteristics.

A 5% v/v stock solution of phosphate buffered saline (PBS) was prepared by mixing 50 mL PBS with 950 mL deionised water. Colloidal biopolymer solutions of 1% w/v concentration were then prepared by mixing 1 g of one of the following biopolymers with a 100 mL aliquot of the stock solution:
(a) alginate;
(b) pectin;
(c) iota-carrageenan;
(d) kappa-carrageenan;
(e) gellan
(f) dextran.

Several drops of black ink were added to aliquots of the colloidal biopolymer solutions and mixed to homogenously disperse the colorant. 1 mL samples of the coloured solutions were then loaded into an airbrush (750 μm aperture) set to 1 bar, and sprayed at a sheet of acetate propped up at a 45° angle in front of the air brush. The airbrush was cleaned between samples using a succession of ethanol and water.

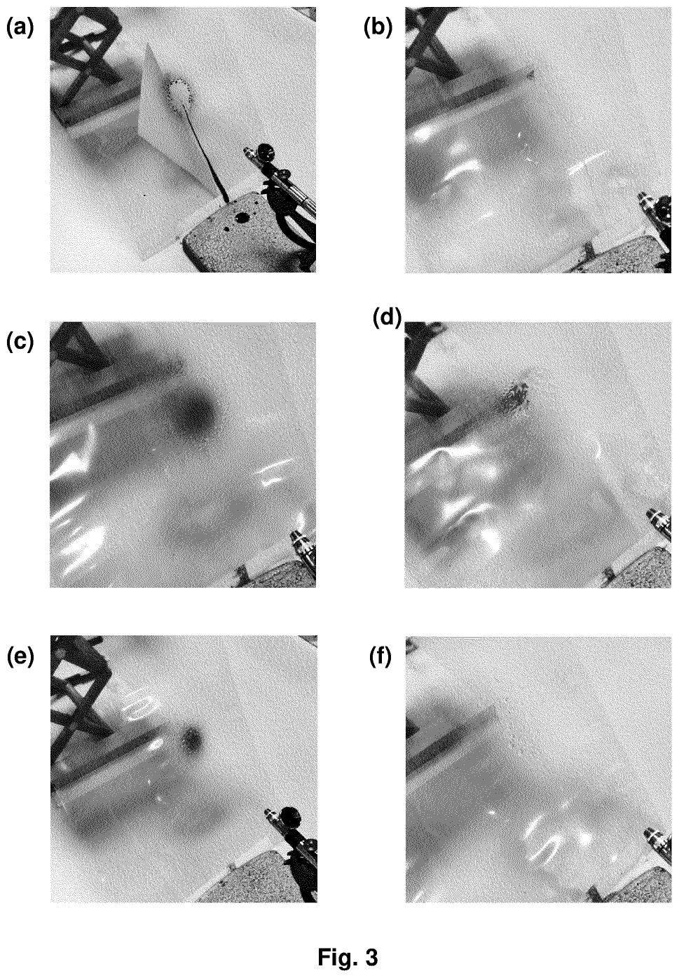

The results of the spray screening are shown in FIG. 3. The formulations comprising alginate, pectin or dextran (a, b and f) appeared to be jettable rather than forming a plume and flowed off of the acetate under their own mass. The formulations comprising gellan or carrageenan (c, d and e), on the other hand, showed good sprayability and retention on the acetate, creating a relatively even coverage at the spray site. Gellan and carrageenan were therefore selected for further study.

Rheology

Viscosity curves for formulations containing the biopolymers used in the initial screening described above, as well as formulations comprising various different combinations of gellan, iota-carrageenan, lambda-carrageenan and mixtures thereof were measured using a rotational rheometer (Kinexus Ultra, Netzsch Geratebeu GmbH, DE) fitted with a cone and plate (4°, 40 mm diameter) geometry. Tests were conducted at 25° C., under stress control. Dynamic viscosity was analysed by reduction of the shear stress from a maximum of 100 to 0.001 Pa (dependent on test material to prevent expulsion from the gap at lower viscosities) over a 2 minute ramp time. Kinexus software was used to characterise the flow profiles using both power law and Cross models.

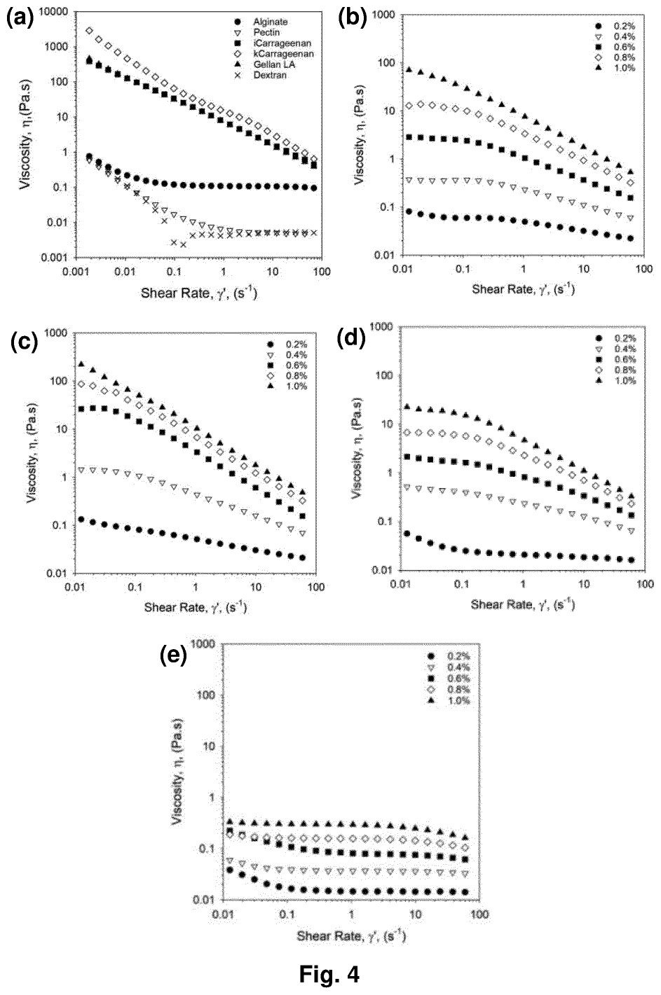

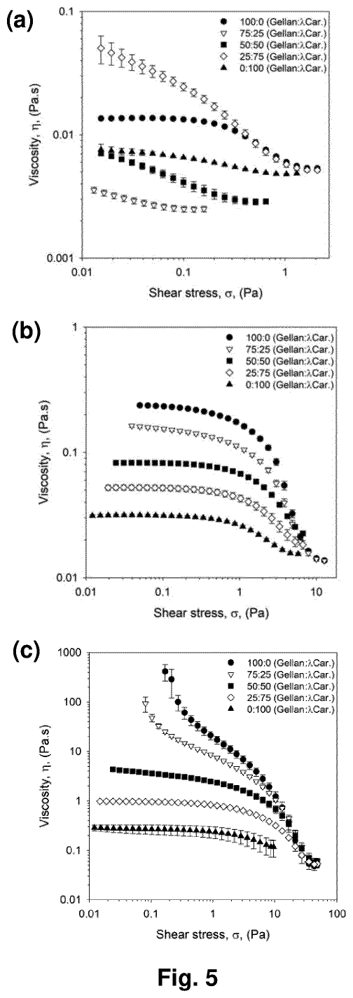

The results are shown in FIGS. 4-5 and Table 1. FIGS. 4(a)-(e) show viscosity data for the formulations comprising the following polymers:
(a) the range of biopolymers used in the initial screening (1% w/v);
(b) iota-carrageenan (0.2-1.0% w/v);
(c) a 50:50 mixture of iota-carrageenan and gellan (0.2-1.0% w/v);
(d) gellan (0.2-1.0% w/v); and
(e) lambda-carrageenan (0.2-1.0% w/v).

FIGS. 5(a)-(c) show viscosity data for varying ratios of gellan and lambda-carrageenan at: (a) 0.2% w/v, (b) 0.4% w/v, and (c) 1.0% w/v.

TABLE 1

| | | Zero-Shear | Consistency | |
|---|---|---|---|---|
| Total Polymer | Polymer Ratio | Viscosity | Coefficient | Rate Index |
| (% (w/v)) | (Gellan:λ-Car.) | (Pa · s) | (K) | (n) |
| 1.0 | 100:0 | N/A | 3.544 ± 0.319 | 0.403 ± 0.004 |
| | 75:25 | N/A | 2.693 ± 0.075 | 0.491 ± 0.002 |
| | 50:50 | 4.080 ± 0.324 | 1.163 ± 0.034 | 0.543 ± 0.005 |
| | 25:75 | 0.988 ± 0.013 | 0.094 ± 0.002 | 0.727 ± 0.012 |
| | 0:100 | 0.274 ± 0.046 | 0.030 ± 0.009 | 0.821 ± 0.151 |
| 0.4 | 100:0 | 0.245 ± 0.002 | 0.065 ± 0.004 | 0.831 ± 0.003 |
| | 75:25 | 0.172 ± 0.005 | 0.060 ± 0.001 | 0.692 ± 0.054 |
| | 50:50 | 0.083 ± 0.000 | 0.020 ± 0.002 | 1.021 ± 0.084 |
| | 25:75 | 0.052 ± 0.002 | 0.016 ± 0.000 | 1.134 ± 0.029 |
| | 0:100 | 0.032 ± 0.001 | 0.013 ± 0.001 | 1.293 ± 0.051 |
| 0.2 | 100:0 | 0.014 ± 0.000 | 0.021 ± 0.000 | 1.315 ± 0.161 |
| | 75:25 | 0.009 ± 0.000 | 0.022 ± 0.000 | 1.413 ± 0.025 |
| | 50:50 | 0.010 ± 0.000 | 0.017 ± 0.003 | 1.341 ± 0.287 |
| | 25:75 | 0.007 ± 0.000 | 0.016 ± 0.007 | 1.283 ± 0.403 |
| | 0:100 | 0.007 ± 0.000 | 0.026 ± 0.001 | 1.288 ± 0.056 |

Flow behaviours for the 1% w/v gellan/lambda-carrageenan systems showed a transition from material characteristics indicative of gellan (viscosity asymptoting at low stresses), to those of the lambda-carrageenan (plateaued viscosities at low stresses), as the ratio of the two polymers shifted from one extreme to the other (100% gellan to 100% lambda-carrageenan). Loss of overall viscosity was observed as the systems shifted from high to low gellan ratios, confirmed by the reduction in consistency coefficient (K) from 3.54 to 0.03. This correlated with the increase in rate index (n), where more gellan resulted in higher degrees of shear thinning: 0.40 for 100% gellan compared to 0.82 for 100% lambda-carrageenan. A reduction in the total polymer content to 0.4% (w/v) resulted in all mixtures being characterised by the Cross model, consistent with data provided for the isolated polymers. Further reduction in the polymer concentration to 0.2% (w/v) resulted in profiles independent on the ratio of gellan to lambda-carrageenan, with samples indistinguishable from each other (within a margin of error).

Viscosity data was also used to better understand the potential residence of the spray within the nasal cavity. Equation 3 was used to predict the stress exerted on the material under gravity residing on an incline.

σₘₐₓ=ρ.g.h.(sinθ) [3]

Where p is the density of the nasal spray (kg.m⁻³), g is the force due to gravity (9.807 m.s⁻²), h is the thickness of the sprayed layer (m) and e is the inclined angle. Applying values for the polymer suspensions based on a maximum 500 μm thick sprayed layer at 45° (Equation 4) resulted in a theoretical stress of 7 mPa.

σₘₐₓ=1.01×9.807×1×10⁻³(sin 45) [4]

A simple force balance revealed insufficient stress under gravity to induce flow in any of the systems containing a dynamic yield stress. Indeed, even in systems described by the Cross model, the external stress due to gravity was not sufficient to move the system from its zero-shear plateau into the thinning region.

Sprayability

The spray behaviour of gellan and lambda-carrageenan was assessed. The test formulations were mixed with black dye (0.1% v/v) and thoroughly shaken to provide a homogeneous mixture. The coloured formulations were then sprayed vertically upwards onto a horizontal paper substrate using a typical handheld spray applicator (Adelphi, UK). The sprayed substrates were allowed to dry in air and scanned at 600 DPI (greyscale). The image files were processed using an image package (ImageJ), where they were initially cropped to a 2000×2000 pixel box visually centred around the spray pattern. Standard thresholding was applied to all images, and the scale was corrected equating 2000 pixels to 100%. Droplet analysis was conducted, and total coverage determined as a percentage of the whole image. Distributions were recorded as x/y co-ordinates and plotted relative to the central droplet.

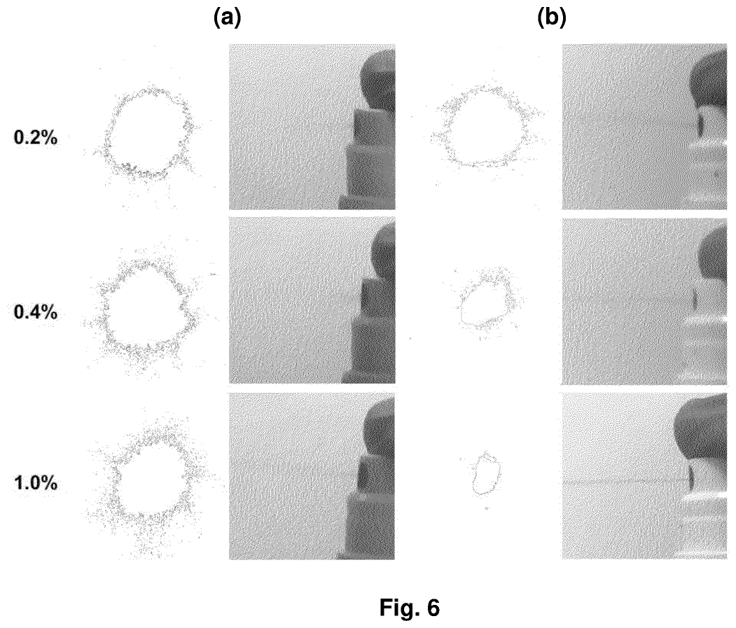

The results are shown in FIGS. 6-9 and Table 2.

TABLE 2

| | Total | Polymer | | Distribution | Spray |
|---|---|---|---|---|---|
| Formulation | Polymer | Ratio | Coverage | radius | angle |
| (A, B) | (% (w/v)) | (A:B) | (%) | (cm) | (°) |
| [A] | 1.0 | 100:0 | 20.7 | 2.2 | 12.2 |
| Gellan | | 75:25 | 9.1 | 1.4 | 8.2 |
| [B] | | 50:50 | 3.6 | 0.9 | 5.2 |
| Lambda- | | 25:75 | 2.7 | 0.8 | 4.5 |
| Carrageenan | | 0:100 | 6.2 | 1.2 | 6.8 |
| | 0.4 | 100:0 | 28.5 | 2.5 | 14.3 |
| | | 75:25 | 22.2 | 2.2 | 12.6 |
| | | 50:50 | 15.3 | 1.9 | 10.6 |
| | | 25:75 | 12.6 | 1.7 | 9.6 |
| | | 0:100 | 8.1 | 1.4 | 7.7 |
| | 0.2 | 100:0 | 26.0 | 2.4 | 13.7 |
| | | 75:25 | 24.8 | 2.4 | 13.4 |
| | | 50:50 | 25.5 | 2.4 | 13.5 |
| | | 25:75 | 20.7 | 2.2 | 12.2 |
| | | 0:100 | 15.9 | 1.9 | 10.7 |
| [A] | 1.0 | 100:0 | 20.7 | 2.2 | 12.2 |
| Gellan | | 75:25 | 25.0 | 2.4 | 13.4 |
| [B] | | 50:50 | 12.0 | 1.7 | 9.4 |
| Iota- | | 25:75 | 8.0 | 1.3 | 7.6 |
| Carrageenan | | 0:100 | 2.8 | 0.8 | 4.5 |
| | 0.4 | 100:0 | 28.5 | 2.5 | 14.3 |
| | | 75:25 | 25.4 | 2.4 | 13.5 |
| | | 50:50 | 18.8 | 2.1 | 11.7 |
| | | 25:75 | 24.3 | 2.3 | 13.2 |
| | | 0:100 | 27.4 | 2.5 | 14.0 |
| | 0.2 | 100:0 | 26.0 | 2.4 | 13.7 |
| | | 75:25 | 25.0 | 2.4 | 13.4 |
| | | 50:50 | 29.0 | 2.6 | 14.4 |
| | | 25:75 | 29.4 | 2.6 | 14.5 |
| | | 0:100 | 22.7 | 2.3 | 12.8 |
| [A] | 1.0 | 100:0 | 17.7 | 2.0 | 11.3 |
| Alginate | | 75:25 | 14.5 | 1.8 | 10.3 |
| [B] | | 0:100 | 2.8 | 0.8 | 4.5 |
| Iota- | | | | | |
| Carrageenan | | | | | |
| [A] | 1.0 | 100:0 | 2.2 | 0.7 | 4.0 |
| Xanthan | | 25:75 | 3.4 | 0.9 | 5.0 |
| [B] | | 0:100 | 2.8 | 0.8 | 4.5 |
| Iota- | | | | | |
| Carrageenan | | | | | |

At 1% w/v total polymer concentration, gellan and alginate showed good sprayability (>13% coverage, >1.8 cm distribution radius and >10° spray angle), while iota-carrageenan, lambda-carrageenan and xanthan were not sprayable. In general, decreasing the total polymer concentration was found to improve sprayability, with formulations being significantly more sprayable than 1.0% formulations, and 0.2% formulations being slightly more sprayable than 0.4% formulations. Adding gellan or alginate was also found to improve the sprayability of iota- and lambda-carrageenan.

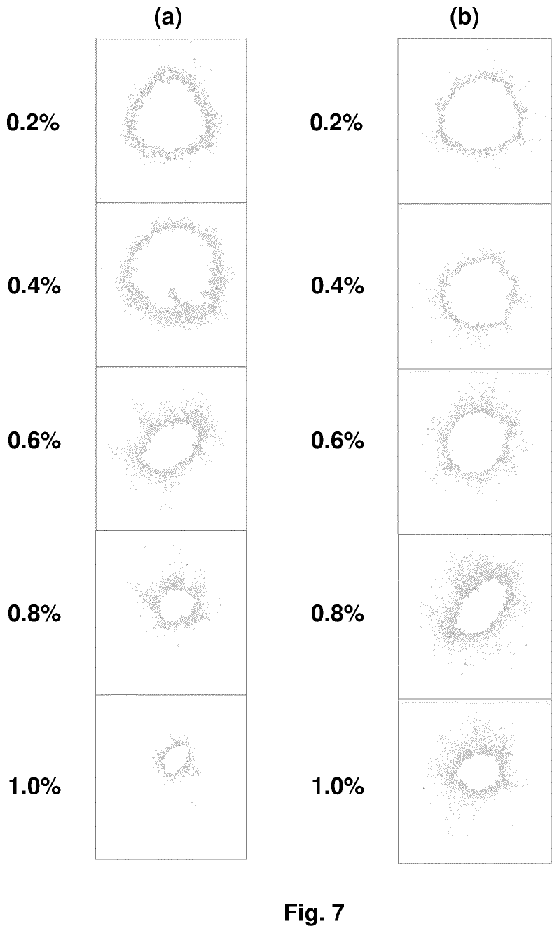

FIGS. 6(a)-(b) show spray distributions for formulations comprising: (a) 100% gellan, and (b) 100% lambda-carrageenan. FIGS. 7(a)-(b) show spray distributions for formulations comprising: (a) 100% iota-carrageenan, and (b) a 50:50 mixture of iota-carrageenan and gellan.

Gellan demonstrated an inherent sprayability, forming a typical “plume” across all concentrations studied. In contrast, lambda-carrageenan systems formed a plume at lower concentrations, but demonstrated an increasing degree of “jetting” at higher concentrations. Iota-carrageenan systems formed a plume at higher concentrations than lambda-carrageenan, but again demonstrated an increasing degree of jetting at higher concentrations. Systems comprising a mixture of iota-carrageenan and gellan demonstrated increased plume formation and a lower degree of jetting at higher concentrations than iota-carrageenan alone.

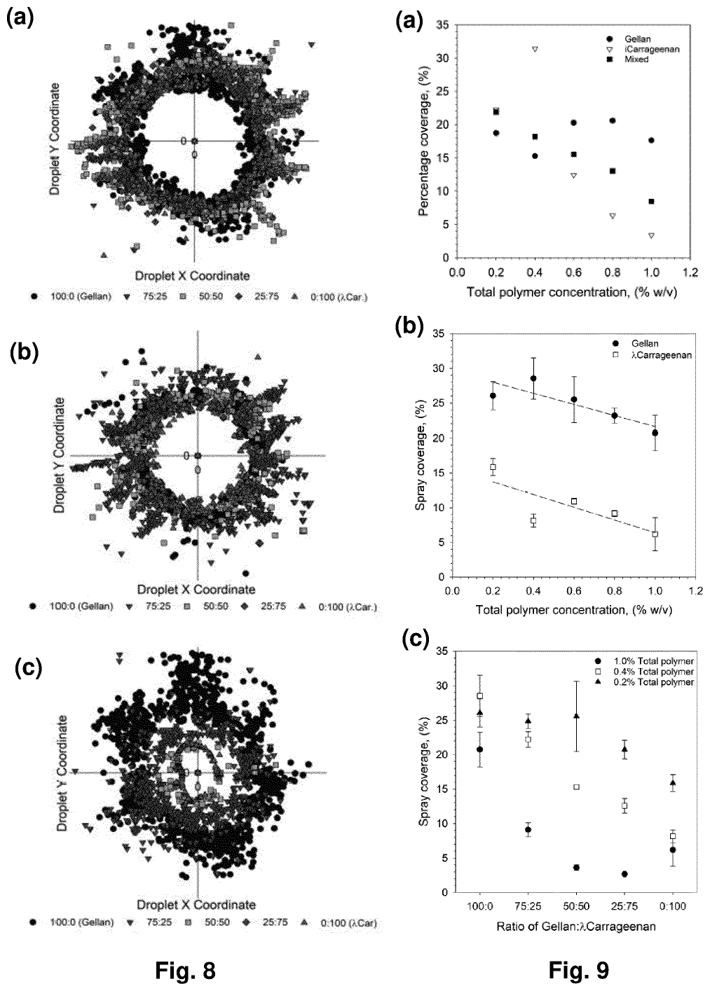

Droplet distributions for mixtures of gellan and lambda-carrageenan at (a) 0.2% (w/v), (b) 0.4% (w/v), and (c) 1% (w/v) total polymer are shown in FIG. 8. As the total polymer content increased, distributions became narrower with fewer satellite droplets forming around the central accumulation.

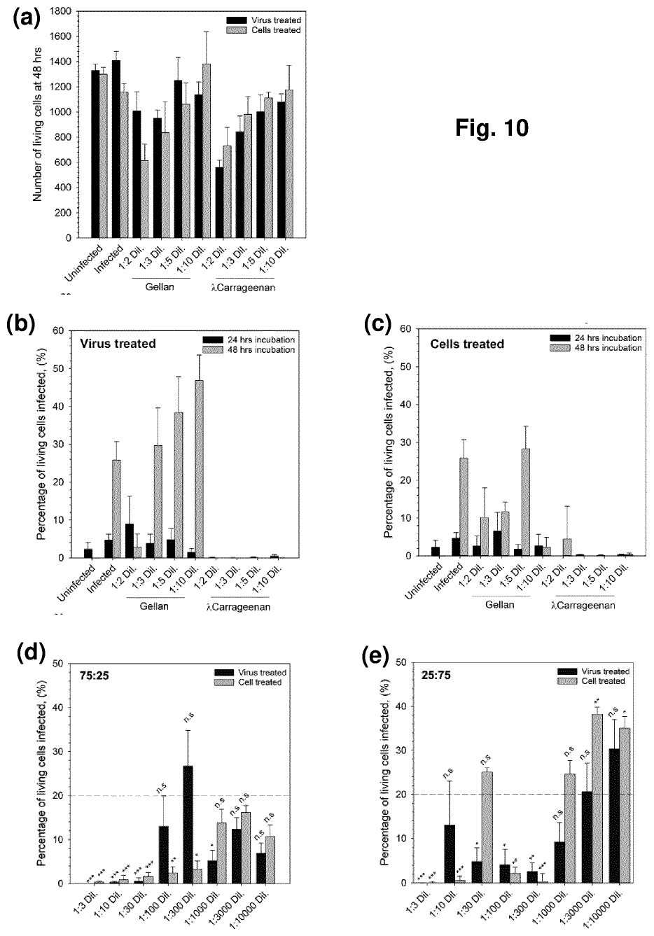

A general negative correlation between percentage coverage and total polymer concentration was drawn, loosely fitting a linear trend (R²=0.72 and 0.62 for gellan and lambda-carrageenan, respectively), as shown in FIG. 9(b). In general, it was observed that all gellan concentrations resulted in higher coverage than lambda-carrageenan alone, demonstrating maximum and minimum coverage of 28.5-20.7% compared to 15.9-6.1% for the lambda-carrageenan systems (0.2 and 1.0% (w/v) polymer, respectively).

The role that total polymer content and ratio of polymers play within the sprayability of composite systems can be clearly seen in FIG. 8. In all instances, irrespective of total polymer concentration, a shift to smaller distributions was observed as the ratio of gellan to lambda-carrageen decreased. These changes became more pronounced when varying the total polymer content, where the magnitude of change between 100% gellan to 100% lambda-carrageenan followed the trend 1.0%>0.4%>0.2% (w/v) total polymer content.

Such observations were mirrored in the total coverage data shown in FIG. 9(b). Replacing 25% of the total lambda-carrageenan with gellan resulted in a 4.9% and 4.4% increase in coverage for the 0.2% and 0.4% (w/v) systems, respectively, with an initial loss in spray coverage (−3.5%) for the 1% total polymer content. Increasing the ratio of gellan:λ-carrageenan to 75:25 increased coverage by 9.0%, 14.1% and 2.9% for the 0.2, 0.4 and 1.0% (w/v) systems, respectively.

In Vitro Analysis

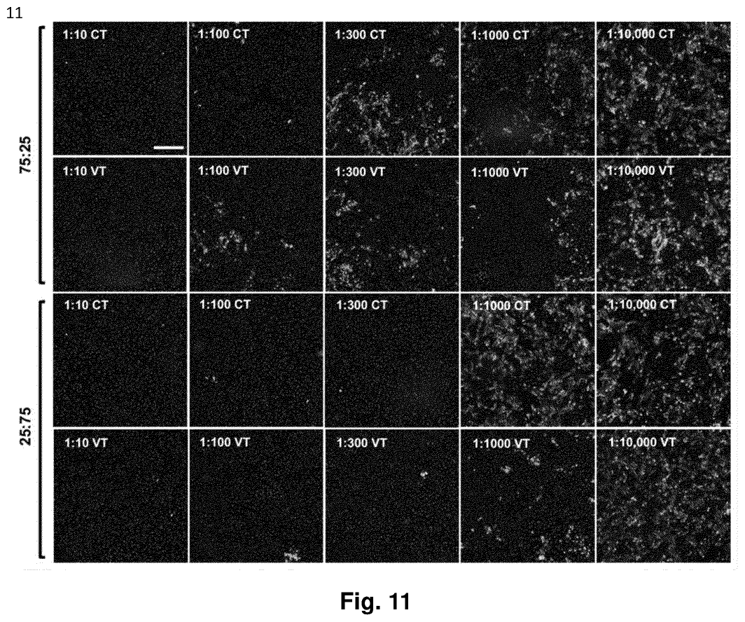

A study was undertaken to determine cell compliance with the spray formulations over a 48 hour testing period. The results are shown in FIG. 10(a). Cell tolerance was found to be dependent on the polymer concentration, demonstrating a 2-fold reduction in the number of living cells for both the gellan and the lambda-carrageenan at a dilution of 1:2. Dose-response was linear (R²=0.96 and 0.97 for gellan and lambda-carrageenan, respectively), with reduced cell death as the systems became increasing more dilute.

Prevention of contraction and/or transmission of coronavirus was assessed using a SARS-CoV-2 assay. Vero cells were seeded in culture media, then infected with SARS-CoV-2 the following day. The formulations were applied using two treatment regimens: treating the virus with the formulation prior to infecting the cells (referred to as virus treated, VT), or by treating the cells with formulation before introduction of the virus (referred to as cells treated, CT). Infection was terminated by cell fixation after 24 or 48 hours, and the number of infected cells was estimated by an algorithm using a CX5 High Content microscope.

FIGS. 10(b) and 10(c) show the effect of single polymer systems on resultant infection under the virus-treated and cell-treated regimens, respectively. It was observed that in the case of the gellan formulations, all dilutions resulted in infection after 24 hours, irrespective of treatment regime. Infection levels were exacerbated after 48 hrs, with all dilutions greater than 1:3 resulting in levels of infection above the control. The lambda-carrageenan systems showed no signs of infection above the uninfected control at either time point, 24 or 48 hrs, irrespective of the treatment regimen.

Composite systems containing 1% total polymer at either a ratio of 75:25 or 25:75 (gellan:iota-carrageenan) were also studied using the same treatment regimens over 48 hours. The results are shown in FIGS. 10(d) and 10(e). Under the cell-treated regimen, composites at a ratio 75:25 showed significant suppression of infection (minimum of p<0.05) up to a dilution of 1:10000, in comparison with the untreated control group (FIG. 9(d)). By contrast, composites at a ratio of 25:75, comprising a higher proportion of iota-carrageenan, demonstrated inconsistent suppression of infection, with dilutions of 1:30, 1:1000, 1:3000 and 1:10000 all resulting in infection levels equal to or greater than the untreated control (FIG. 9(e)).

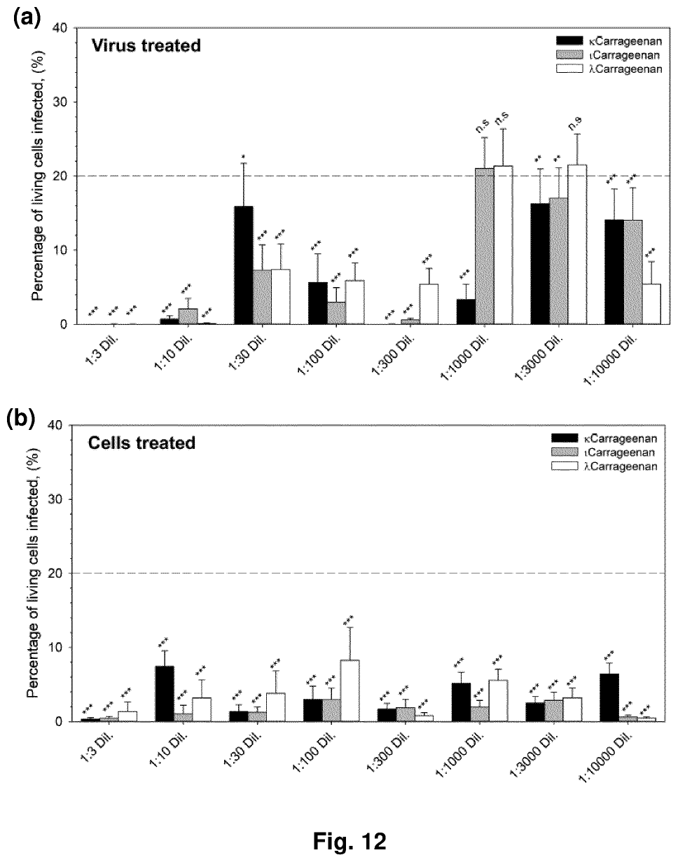

Comparison of the treatment regimens highlighted differences in the ability to supress infection. For the 25:75 composite, treating the cells first was more effective at lower dilution factors of between 1:3 and 1:300 (with the exception of 1:30) compared to treating the virus first. However, at larger dilution factors (>1:300) treatment of the virus first became more effective. For the 75:25 composite, treating the virus first was more effective at suppressing infection at all dilutions, except at dilutions of 1:100 and 1:300, where treating the cells first was more effective. This can be seen more clearly in the Hoechst stained images (FIG. 11), where the extent of infected cells (light-coloured) was generally lower for the virus treated groups when compared to the cell treated groups.

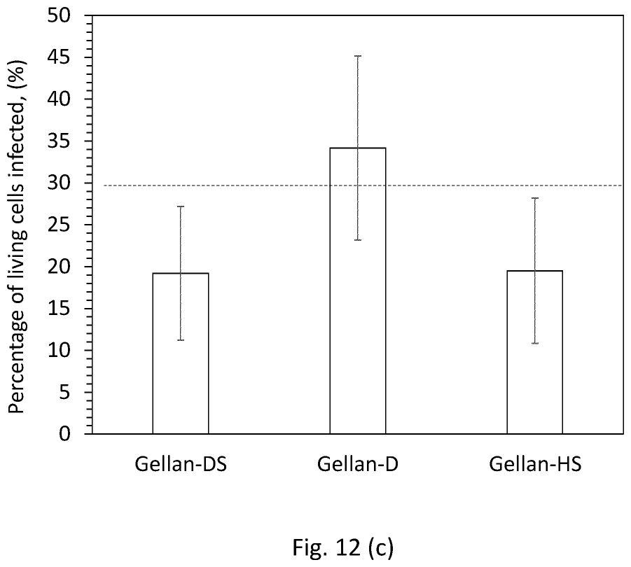

In order to determine whether the degree of sulphation across the polymer backbone was important in order to suppress infection, kappa-, iota- and lambda-carrageenan were studied using the SARS-CoV-2 assay, under virus-treated and cells-treated regimens, for 48 hours (see FIGS. 12(a) and (b)).

It was observed that in all cases where the cells were treated first, infection was significantly reduced compared to the untreated control group (p<0.001). This could not be said for the virus-treated regimen, where higher dilution factors (1:1000 and 1:3000) did not statistically affect the degree of infection for both the iota- and lambda-carrageenan formulations. Furthermore, no correlation could be drawn between the degree of sulphation of the polymer and its ability to supress infection.

In a related experiment, various polymer mixtures were tested: Gellan-Dextran Sulphate (DS), Gellan-Dextran (D), Gellan-Heparan Sulphate (HS). As shown in FIG. 12(c), the addition of sulphated groups reduces the level of infection in comparison to treatments without a sulphated polysaccharide (dotted line in FIG. 12c shows the level of infection for Gellan only systems).

Cell Binding Study

Gellan and carrageenans were assessed for their ability to bind to human cells. Vero cells were expanded in T75 flasks, washed with PBS (5 ml) and removed using TrypLE (2.5 ml). The cells were then re-suspended in complete media and seeded into well plates (10,000 cells per well). Cells were left to attach over the subsequent 24 hrs prior to treatment. Cells were then washed three times with PBS and the final washing was removed. The polymer formulations were diluted by a factor of 1:3 or 1:5 and placed over the cells (200 μl); controls were treated with equal volumes of PBS. Cells were incubated for 30 minutes prior to washing (three times) with PBS. Cells were subsequently stained with Alcian blue (0.1%) for 30 minutes, before a final wash in PBS to remove residual stain. PBS was then added (200 μl) and the wells were imaged using a Cytation 5M automated microplate imager, in bright field using a x4 optical lens focused on the centre of each well. Wells were divided into a 6×4 matrix and stitched together retrospectively. Images were then cropped to the well diameter using a software package (ImageJ) and the colour thresholding was standardised, then analysed for mean intensity.

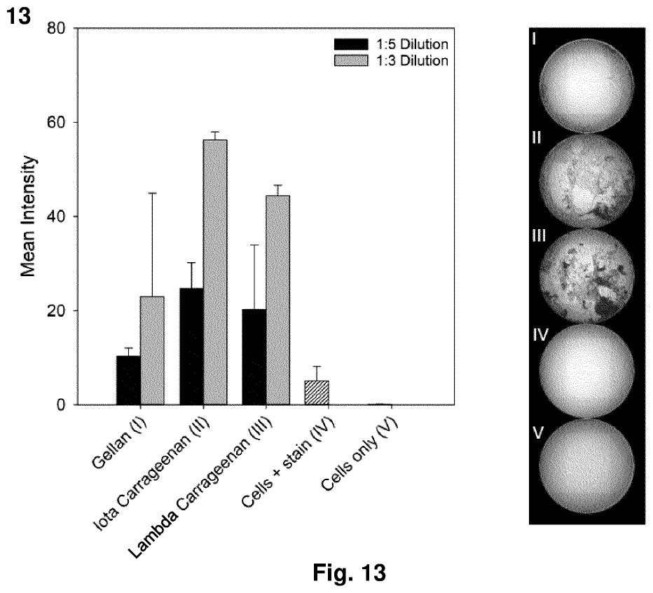

The results are shown in FIG. 13, wherein the wells were treated with the following:
(I) gellan;
(II) iota-carrageenan;
(Ill) lambda-carragenan;
(IV) cells and stain only (control);
(V) cells only (control).

The intensity data showed a significant difference (p<0.001) between cells treated with a 1:3 dilution of both carrageenans when compared to the cells only group. Moreover, when compared to the stained cells only group, significance remained (p<0.01). Gellan did not show significant binding to the cells.

Inter-carrageenan analysis demonstrated iota-carrageenan to have a higher mean intensity in comparison to lambda-carrageenan (56.2% and 44.4%, respectively). However, the lambda-carrageenan sample appeared to show areas of higher maximum intensity in comparison to the iota-carrageenan.

Viral Transfection Study

4 categories were tested (Gellan, Gellan-Dextran, Gellan-Dextran S and Gellan-λ Carrageenan) as well as Controls, which contained an equivalent volume of medium instead of the gel formulation.

Human primary osteoblastic cells at passage 4 were cultured in Dulbecco's Modified Eagle Medium (Thermo Fisher Scientific, USA), containing 10% FCS and 1% Penicillin-Streptomycin (Sigma Aldrich, Germany). They were harvested using Trypsin-EDTA (Lonza, USA) according to standard procedures and were seeded into 6 well dishes at a density of 15.000 cells per well (34.8 mm diameter), in triplicate per category.

Cells were allowed to attach over 24 hours. After this period, the medium was aspirated and 800 μl of culture medium were added, following the addition of 200 μl of each formulation (Gellan, Gellan-Dextran, Gellan-Dextran S and Gellan-λ Carrageenan).

Controls received 200 μl of medium instead of the gel substance.

On contact with the small liquid residue, the gels formed disc pellets, which hovered over the surface of the cells. 1 ml of infected medium was added within each well, which contained viral particles dispersed so that 3.75 μl of the viral load, accounting for viral particles per cell (PPC) were distributed in each well. Cells were allowed to incubate for 18 hours, following which the medium and pellets were aspirated. 2 ml of fresh medium were added and incubated with a nuclear stain, NucBlue Live Cell stain containing Hoechst 33342 (Invitrogen, Life Technologies, Oregon, USA), at a ratio of 2 drops/ml and allowed to incubate for 20-25 minutes before imaging.

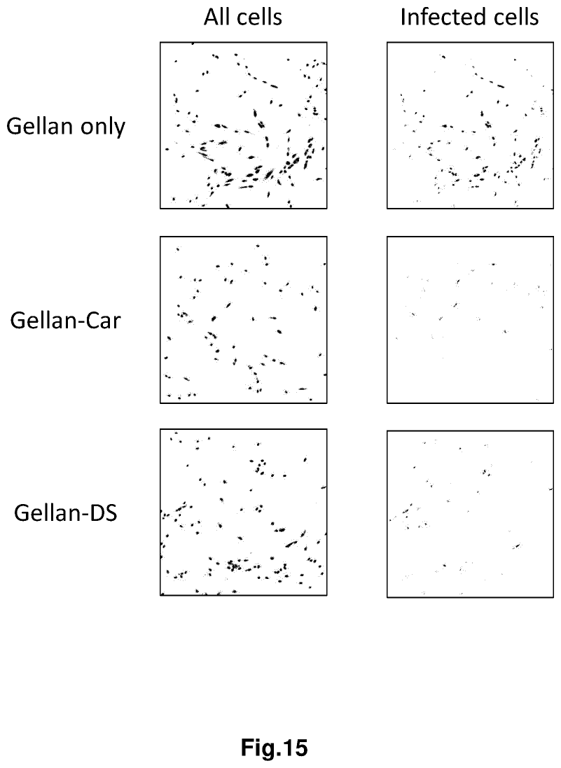

Cells (such as those exemplified in FIG. 15) were imaged using an Olympus Fluoview FV1000 confocal laser scanning microscope (Olympus, Tokyo, Japan). Images acquired from excitation at 405/543 nm wavelengths were collected in individual channels and combined using the Fluoview FV10-ASW software, version 4.2 (Olympus, Tokyo, Japan). Cells were imaged under identical conditions and laser parameters.

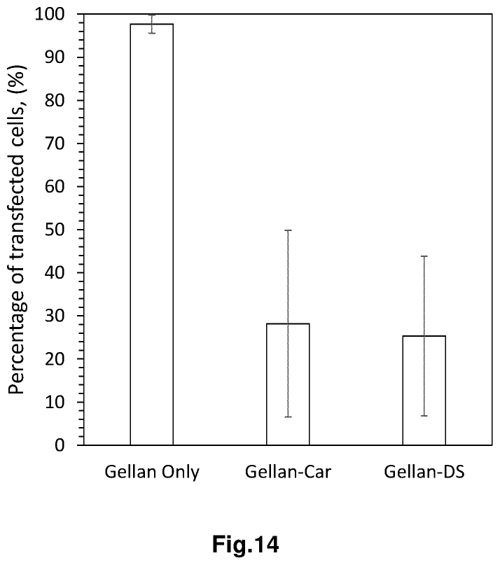

Results are shown in FIG. 14, which shows that in the presence of sulphated polysaccharides (carrageenan and dextran sulphate), the ability of the virus to transfect cells was inhibited in comparison to the gellan only.

1. A sprayable antiviral formulation comprising an antiviral agent and a carrier polymer, wherein the antiviral agent comprises a sulphated polysaccharide and the carrier polymer comprises a non-sulphated polysaccharide.

2. The formulation of claim 1, for use as an oral spray and/or a nasal spray.

3. The formulation of claim 1, for use as a multi-surface spray.

4.-5. (canceled)

6. The formulation of claim 1, wherein the antiviral agent comprises a mucoadhesive sulphated polysaccharide.

7. The formulation of claim 1, wherein the antiviral agent is selected from the group consisting of carrageenans, fucoidans, ulvans, heparan sulphate, heparin, dextran sulphate and mixtures thereof, such as a carrageenan, dextran sulphate, or heparan sulphate.

8. (canceled)

9. The formulation of claim 87, wherein the carrageenan is selected from the group consisting of lambda-carrageenan, iota-carrageenan and kappa-carrageenan.

10. The formulation of claim 1, wherein the formulation has a dynamic viscosity of 0.05 to 100 Pa·s at 25° C.

11. The formulation of claim 1, wherein the formulation further comprises a diluent, such as a saline solution.

12. (canceled)

13. The formulation of claim 1, wherein the concentration of antiviral agent in the formulation is from 0.1 to 1.0% w/v.

14. The formulation of claim 1, wherein the carrier polymer comprises a mucoadhesive non-sulphated polysaccharide.

15. The formulation of claim 1, wherein the carrier polymer is selected from the group consisting of gellan, dextran, alginate, pectin, xanthan, and mixtures thereof, such as gellan.

16. (canceled)

17. The formulation of claim 14, wherein the formulation has a total polymer concentration consisting of, or consisting essentially of, the concentration of antiviral agent and the concentration of carrier polymer in the formulation.

18. The formulation of claim 17, wherein the total polymer concentration is from 0.1 to 2.0% w/v, such as from 0.1 to 1.0% w/v.

19. (canceled)

20. The formulation of claim 14, wherein the ratio of antiviral agent to carrier polymer is from 90:10 to 10:90, such as from 10:90 to 50:50.

21. (canceled)

22. The formulation of claim 1, further comprising a dispersing agent.

23. The formulation of claim 1, wherein the formulation is free or substantially free of oxidising agents.

24. A spray device comprising:
the formulation of claim 1;
a body for containing the formulation therein; and
a nozzle for spraying the formulation.

25. The spray device of claim 24, wherein the spray device is a nasal spray device.

26. A nasal spray formulation comprising an antiviral agent and a carrier polymer, wherein:
the antiviral agent is a sulphated polysaccharide, and
the carrier polymer is a non-sulphated polysaccharide.

27. A method of preventing infection or transmission of coronavirus such as SARS-CoV-2, the method comprises administering the formulation of claim 1 to a subject in need thereof, or spraying a formulation of claim 1.
